(19) United States
(12) Patent Application Publication
(10) Pub. No.: US 2002/0155093 A1
(43) Pub. Date: Oct. 24, 2002

(54) Method for stimulation of an immune response to a differentiation antigen stimulated by an altered antigen

(76) Inventors: Alan N. Houghton, New York, NY (US); Clariss Naftzger, San Carlos, CA (US); Vijayasaradhi Setaluri, Winston-Selem, NC (US)

Correspondence Address: OPPEDAHL AND LARSON LLP, P O BOX 5068, DILLON, CO, 80435-5068, US

(21) Appl. No.: 10/041,410

(22) Filed: Jan. 7, 2002

Related U.S. Application Data
(62) Division of application No. 09/627,694, filed on Jul. 28, 2000.
Application No. 09/627,694 is a continuation-in-part of application No. 09/308,697, filed on May 21, 1999, now Pat. No. 6,328,969.
Application No. 09/308,697 is a 371 of international application No. PCT/US97/22669, filed on Dec. 10, 1997.
(60) Provisional application No. 60/036,419, filed on Feb. 18, 1997.

(51) Int. Cl. A61K 39/00; A61K 38/19

(52) U.S. Cl. 424/85.1; 424/184.1; 424/277.1

(57) ABSTRACT

Tolerance of the immune system for self differentiation antigens can be overcome and an immune response stimulated by administration of a therapeutic differentiation antigen. The therapeutic differentiation antigen is altered with respect to the target differentiation antigen in the individual being treated (i.e., the differentiation antigen to which an immune response is desired) in one of three ways. First, the therapeutic differentiation antigen may be syngeneic with the target differentiation antigen, provided that therapeutic differentiation antigen is expressed in cells of a species different from the individual being treated. For example, a human differentiation antigen expressed in insect or other non-human host cells can be used to stimulate an immune response to the differentiation antigen in a human subject. Second, the therapeutic differentiation antigen may be a mutant form of a syngeneic differentiation antigen, for example a glycosylation mutant. Third, the therapeutic differentiation antigen may be a differentiation antigen (wild-type or mutant) of the same type from a species different from the individual being treated. For example, a mouse differentiation antigen can be used to stimulate an immune response to the corresponding differentiation antigen in a human subject. Administration of altered antigens in accordance with the invention results in an effective immunity against the original antigen expressed by the cancer in the treated individual.

This application is a divisional of U.S. patent application Ser. No. 09/627,694, filed Jul. 28, 2000, which is a continuation-in-part of U.S. patent application Ser. No. 09/308,697, filed May 21, 1999, now U.S. Pat. No. 6,328,969, which is a §371 National Phase of International Application No. PCT/US97/22669 filed Dec. 10, 1997. The application also claims the benefit under 35 USC §119(e) of U.S. Provisional Application No. 60/036,419 filed Feb. 18, 1997, all of which are incorporated herein by reference.

### FIELD OF THE INVENTION

This application relates to a method and compositions for stimulation of an immune response to differentiation antigens.

### BACKGROUND OF THE INVENTION

Most tumor immunity is mediated by recognition of self-antigens, antigens present in cancer cells that are also found in normal host tissue. Houghton, A. N., J. Exp. Med. 180: 1-4 (1994). This type of immunity is more akin to autoimmunity than to immunity in infectious diseases, where the response is directed at a truly foreign antigen, present in the pathogen but not in host tissue. Evidence of this can be found in the autoimmune sequelae that often follow the development of successful tumor immunity. Bowne, W. B., et al., J. Exp. Med. 190(11):1717-1722 (1999).

Differentiation antigens form one prototype of self-antigens in cancer immunity. Houghton, A. N., et al., J. Exp. Med. 156(6):1755-1766 (1982). Differentiation antigens are tissue-specific antigens that are shared by autologous and some allogeneic tumors of similar derivation, and on normal tissue counterparts at the same stage of differentiation. Differentiation antigens have been shown to be expressed by a variety of tumor types, including melanoma, leukemia, lymphomas, colorectal, carcinoma, breast carcinoma, prostate carcinoma, ovarian carcinoma, pancreas carcinomas, and lung cancers. Typically the expression of these antigens changes as a cell matures and can characterize tumors as more or less differentiated. For example, differentiation antigens expressed by melanoma cells include Melan-A/MART-1, Pmel17, tyrosinase, gp75 and gp100. Differentiation antigens expressed by lymphomas and leukemia include CD19 and CD20/CD20 B lymphocyte differentiation markers. An example of a differentiation antigen expressed by colorectal carcinoma, breast carcinoma, pancreas carcinoma, prostate carcinoma, ovarian carcinoma, and lung carcinoma is the mucin polypeptide muc-1. A differentiation antigen expressed by breast carcinoma is her2/neu. The her2/neu differentiation antigen is also expressed by ovarian carcinoma. Differentiation antigens expressed by prostate carcinoma include prostate specific antigen, prostatic acid phosphatase, and prostate specific membrane antigen (PSMA).

Immune recognition of human cancer has been intensively investigated in melanoma, a cancer arising from pigment cells (melanocytes) in the skin. Houghton, A. N., J. Exp. Med. 180: 1-4 (1994). Studies of melanoma have surprisingly shown that the immune system commonly recognizes products of genes that are specifically expressed by melanocytes, particularly genes that are involved in synthesis of pigment. Houghton, A. N., J. Exp. Med. 180: 1-4 (1994); Sakai, C., et al, Melanoma Res. 7: 83-95 (1997). Examples include tyrosinase, the critical enzyme required for synthesis of the pigment melanin, and tyrosinase-related proteins (TRP) that determine the type of melanin synthesized (TRP-2 and gp75^{TRP-1}). Vijayasaradhi, S., et al., J. Exp. Med. 171:1375-80 (1990); Bouchard, V., et al., J. Exp. Med. 169: 2029-2042 (1989).

Some patients with melanoma demonstrate both T-cell and antibody mediated recognition of several members of this family of antigens. gpl 100, a structural component of the melanosome, was identified as an antigen recognized by patients in some of these studies. Monoclonal antibodies against differentiation antigens such as HMB-45 (specific for gp100) are a commonly applied immunohistochemical technique utilized in the diagnosis of melanoma. Melanosomal antigens including TRP-2, gp75^{TRP-1} and gp100 have also been defined as tumor rejection antigens in C57BL/6 mice.

TRP-2, gp75^{TRP-1}, and gp100 provide murine models for differentiation antigens with relevance to a human cancer. Med. 178: 489-95 (1993); Wang, R. F., et al., J. Exp. Med. 184: 2207-2216 (1996).

These melanoma/melanocyte differentiation antigens have been shown to be recognized by autoantibodies and T cells of persons with melanoma, and to be relevant autoantigens. Wang et al., J. Exp. Med. 183: 799-804 (1996); Vijayasaradhi et al., J. Exp. Med. 171: 1375-1380 (1990); Topalain, S. L. et al., Proc. Natl. Acad. Sci. USA, 91:9461-9465 (1994). In patients with metastatic melanoma, CD8⁺lymphocytes have been detected that are specific for human gp100 (hgp100). Riker, A., et al., Surgery 126(2):112-120 (1999); Kammula, U. S., et al., J. Immunol. 163(12):6867-6875 (1999). The mouse counterpart of gp100 (mgp100) is the product of the silver locus and is also known as pmel17. Mgp100 is 76% identical at the amino acid level to hgp100. Zhai, Y., et al., J. Immunother. 20(1):15-25 (1997).

While immunity against some viruses can develop in the absence of CD4⁺ T-cell help, to our knowledge, tumor immunity against differentiation antigens has never been shown to arise independently of (CD4⁺ T-cells. Help from CD4⁺ T-cells is though to be necessary to prime the antigen presenting cell in the induction of immunity or in the secretion of cytokines that may facilitate tumor cell destruction in the effector phase of the immune response. Similarly, CD4⁺ T-cells are thought to be essential in the pathogenesis of many autoimmune diseases. Kumar, V., et al., J. Exp. Med. 184(5):1609-1617 (1996); De Silva, H. D., Immunology 93(3):405-408 (1998). We have previously reported that xenogeneic immunization of C57BL/6 mice with the melanosomal antigen gp75^{TRP-1} induces tumor immunity and autoimmunity that is mediated by autoantibodies and requires CD4⁺ T-cells. Weber, L. W., et al., J. Clin. Invest. 102(6):1258-1264 (1998). We have also reported that xenogeneic immunization of the same strain of mice with another homologous melanosomal antigen, human TRP-2, results in tumor immunity and autoimmunity that is mediated by T-cells and requires both CD8⁺ and CD4⁺ cell populations. Brown, W. B., et al., J Exp. Med. 190(11):1717-1722 (1999).

Unfortunately, in most cases, the immune system of the individual is tolerant of these antigens, and fails to mount an effective immune response. For the treatment of cancers where the tumor expresses differentiation antigens therefore, it would be desirable to have a method for stimulating an immune response against the differentiation antigen in vivo. It is an object of the present invention to provide such a method.

### SUMMARY OF THE INVENTION

It has now been found that the tolerance of the immune system for self differentiation antigens can be overcome and an immune response stimulated by administration of a therapeutic differentiation antigen. The therapeutic differentiation antigen is altered with respect to the target differentiation antigen in the individual being treated (i.e., the differentiation antigen to which an immune response is desired) in one of three ways. First, the therapeutic differentiation antigen may be syngeneic with the target differentiation antigen, provided that therapeutic differentiation antigen is expressed in cells of a species different from the individual being treated. For example, a human differentiation antigen expressed in insect or other non-human host cells can be used to stimulate an immune response to the differentiation antigen in a human subject. Second, the therapeutic differentiation antigen may be a mutant form of a syngeneic differentiation antigen, for example a glycosylation mutant. Third, the therapeutic differentiation antigen may be a differentiation antigen (wild-type or mutant) of the same type from a species different from the individual being treated. For example, a mouse differentiation antigen can be used to stimulate an immune response to the corresponding differentiation antigen in a human subject. Administration of altered antigens in accordance with the invention results in an effective immunity against the original antigen expressed by the cancer in the treated individual.

Further aspects of the invention are certain compositions and cell lines which are useful in practicing the method of the invention. In particular, the invention includes non-human cell lines, for example insect cell lines, expressing a human differentiation antigen and expression vectors useful in generating such cell lines.

### BRIEF DESCRIPTION OF THE DRAWINGS

FIG. 1 summarizes the results of a tumor protection experiment using mice immunized with human gp75 expressed in Sf9 insect cells; and

FIG. 2 summarizes the results of a tumor protection using mice immunized by gene gun with DNA encoding xenogeneic human gp75.

FIGS. 3A-3D show the protection and rejection of mouse melanoma B16F10/LM3 (B16) after immunization with human TRP-2 but not mouse TRP-2 DNA.

FIGS. 4A-4C show the cytotoxic T cell response to TRP-2₁₈₁₋₁₈₈ peptide in mice that were or were not immunized with mouse TRP-2 or human TRP-2.

FIGS. 5A-5D show the tumor rejection in C57BL/6 mice deficient in immune molecules and cells.

FIG. 6 shows coat depigmentation of a representative mouse treated with human TRP-2 DNA compared to a mouse treated with mouse TRP-2 DNA.

FIG. 7 shows depigmentation in mice treated with human or mouse TRP-2 DNA.

FIGS. 8A and 8B show tumor rejection in perforin deficient mice and gld/gld mice (deficient in fas ligand) treated with human TRP-2 compared to mice not treated.

FIGS. 9A-9E show tumor protection and autoimmunity in mice treated with human gp100.

FIG. 10 shows coat depigmentation in mice treated with human gp100.

FIGS. 11A-11H show results from RMA-S stabilization assays for human gp100 peptide pairs.

FIGS. 12A-12C show CTL responses of mice treated with human and mouse gp100.

FIGS. 13A and 13B show the CD8⁺ T-cell response of mice treated with human or mouse gp100.

FIGS. 14A and 14B show tumor protection in MHC class I and MHC class II deficient mice treated with human gp100.

FIG. 15 shows the CD8⁺ T-cell response of MHC class II deficient mice treated with human gp100.

FIGS. 16A and 16B show the cytotoxic T-cell response to murine tyrosinase of mice immunized with huTYR or muTYR.

FIG. 17 shows tumor protection and rejection of mice immunized with muTYR.

FIG. 18 shows xenogeneic immunization of mice with human PSMA.

### DETAILED DESCRIPTION OF THE INVENTION

The present invention provides a method for stimulating an immune response to a tissue expressing a target differentiation antigen in a subject individual. The subject individual is preferably human, although the invention can be applied in veterinary applications to animal species, preferably mammalian or avian species, as well.

As used in the specification and claims of this application, the term “immune response” encompasses both cellular and humoral immune responses. Preferably, the immune response is sufficient to provide immunoprotection against growth of tumors expressing the target differentiation antigen. The term “stimulate” refers to the initial stimulation of a new immune response or to the enhancement of a pre-existing immune response.

In accordance with the invention, a subject individual is treated by administering a therapeutic differentiation antigen of the same type as the target differentiation antigen in an amount effective to stimulate an immune response. Thus, for example, if the target differentiation antigen is the gp75 antigen found in melanoma cells and melanocytes, the therapeutic antigen is also a gp75 antigen. Differentiation antigens expressed by melanoma cells include Melan-A/MART-1, Pmel17, tyrosinase, gp75 and gp100. Differentiation antigens expressed by lymphomas and leukemia include CD19 and CD20/CD20 B lymphocyte differentiation markers. An example of a differentiation antigen expressed by colorectal carcinoma, breast carcinoma, pancreas carcinoma, prostate carcinoma, ovarian carcinoma, and lung carcinoma is the mucin polypeptide muc-1. A differentiation antigen expressed by breast carcinoma is her2/neu. The her2/neu differentiation antigen is also expressed by ovarian carcinoma. Differentiation antigens expressed by prostate carcinoma include prostate specific antigen, prostatic acid phosphatase, and prostate specific membrane antigen (PSMA). It has been found experimentally, however, that administration of syngeneic differentiation antigens expressed in cells of the same species as the subject individual are not effective for stimulating an immune response (See Examples 1 and 2). Thus, to be effective in the method of the invention, the therapeutic differentiation antigen must be altered relative to the target differentiation.

In a first embodiment of the invention, the therapeutic differentiation antigen and the target are both from the same species. The therapeutic differentiation antigen is produced by expression in cells of a second species different from the first species. In a second embodiment of the invention, the therapeutic differentiation antigen is a mutant form of a syngeneic differentiation antigen. In a third embodiment of the invention, the therapeutic differentiation antigen is a xenogeneic differentiation antigen. Each of these embodiments will be discussed in turn below.

Administration of the therapeutic differentiation antigen can be accomplished by several routes. First, the therapeutic differentiation antigen may be administered as part of a vaccine composition which may include one or more adjuvants such as alum, QS21, TITERMAX or its derivatives, incomplete or complete Freund's and related adjuvants, and cytokines such as granulocyte-macrophage colony stimulating factor (GM-CSF), fit-3 ligand, interleukin-2, interleukin-4 and interleukin-12 for increasing the intensity of the immune response. The vaccine composition may be in the form of a therapeutic differentiation antigen in a solution or a suspension, or the therapeutic differentiation antigen may be introduced in a lipid carrier such as a liposome. Such compositions will generally be administered by subcutaneous, intradermal or intramuscular route. Vaccine compositions containing expressed therapeutic differentiation antigen are administered in amounts which are effective to stimulate an immune response to the target differentiation antigen in the subject individual. The preferred amount to be administered will depend on the species of the target individual and on the specific antigen, but can be determined through routine preliminary tests in which increasing doses are given and the extent of antibody formation or T cell response is measured by enzyme-linked immunosorbent assay (ELISA) or similar tests. T cell responses may also be measured by cellular immune assays, such as cytotoxicity, cytokine release assays and proliferation assays.

The mutant syngeneic or xenogeneic therapeutic differentiation antigen may also be introduced in accordance with the invention using a DNA immunization technique in which DNA encoding the antigen is introduced into the subject such that the antigen is expressed by the subject.

Syngeneic Antigen Expressed in Cells of Different Species

In accordance with the present invention, an immune response against a target differentiation antigen can be stimulated by the administration of syngeneic differentiation antigen expressed in cells of a different species. In general, the subject being treated will be a human or other mammal. Thus, insect cells are a preferred type of cells for expression of the syngeneic differentiation antigen. Suitable insect cell lines includes Sf9 cells and Schneider 2 Drosophila cells. The therapeutic differentiation antigen could also be expressed in bacteria, yeast or mammalian cell lines such as COS or Chinese hamster ovary cells. Host cells which are evolutionarily remote from the subject being treated, e.g. insects, yeast or bacteria for a mammalian subject, may be preferred since they are less likely to process the expressed protein in a manner identical to the subject.

To provide for expression of the differentiation antigen in the chosen system, DNA encoding the differentiation antigen or a portion thereof sufficient to provide an immunologically effective expression product is inserted into a suitable expression vector. There are many vector systems known which provide for expression of incorporated genetic material in a host cell, including baculovirus vectors for use with insect cells, bacterial and yeast expression vectors, and plasmid vectors (such as psvk3) for use with mammalian cells. The use of these systems is well known in the art.

For treatment of humans with a syngeneic differentiation antigen, cDNA encoding the human differentiation antigen to be targeted must be available. cDNA is produced by reverse transcription of mRNA, and the specific cDNA encoding the target differentiation antigen can be identified from a human cDNA library using probes derived from the protein sequence of the differentiation antigen. The cDNA sequences of various human differentiation antigens have been derived by these methods and are known in the art. For example, the sequence of human gp75 (also known as Tyrosinase-related Protein-1 or TRP-1) is known from Vijayasaradhi, S., Bouchard, B., Houghton, A. N., “The Melanoma Antigen Gp75 Is the Human Homologue of the Mouse B (Brown) Locus Gene Product”,. J. Exp. Med. 171: 1375-1380 (1990); Bouchard et al., J. Exp. Med. 169: 2029-2042 (1989). Other human differentiation antigens with known cDNA sequences are gp100 (Kawakami et al, Proc. Nat'l. Acad. Sci. (USA) 91: 6458-6462 (1994); Adema et al., J. Biol. Chem. 269: 20126-20133 (1994)), and mart-1/melan-a for malignant melanoma; CD19 and CD20 for non-Hodgkin's lymphoma; her-2/neu for breast carcinoma (King et al., Science 229: 874-976 (1985); muc-1 for breast, colorectal, lung and pancreatic carcinomas (Spicer et al., J. Biol. Chem. 266: 15099-15109 (1991)); prostate specific membrane antigen (PSMA), prostate specific antigen, and prostatic acid phosphatase for prostate carcinoma (Israeli et al., Cancer Res. 54:

6344-6347 (1994); Monne et al., Cancer Res. 54: 6344-6437 (1994); Vihko et al., FEBS Lett. 236: 275-281 (1988)).

The therapeutic differentiation antigen expressed in cells of a different species is administered to the subject individual in an amount effective to induce an immune response. The composition administered may be a lysate of cells expressing the therapeutic differentiation antigen, or it may be a purified or partially purified preparation of the therapeutic differentiation antigen.

Mutant Forms of Syngeneic Differentiation Antigen

In the second embodiment of the invention, a mutant form of a syngeneic differentiation antigen of a type expressed by the target tumor is used to stimulate an immune response directed against the tumor. For example, if the tumor is a human tumor that expresses gp75, then a mutant form of human gp75 is used as the therapeutic differentiation antigen.

It will be appreciated by persons skilled in the art that not all mutations will produce an antigen which is useful in the method of the present invention. For example, large-scale deletions which eliminate important epitopes would not be expected to work and are not considered to be therapeutic differentiation antigens as that term is used in the specification and claims of this application. Less extensive mutations, however, particularly those which alter the tertiary and/or quaternary structure of the expressed differentiation antigen are within the scope of the present invention.

A preferred type of mutant form of therapeutic differentiation antigen is a glycosylation mutant. On any given membrane protein, there will generally be one or multiple glycosylation sites, with each site being of different importance in its effect on the transport and degradation of the protein. For example, in the case of mouse gp75, there are six N-glycosylation sites, one of which strongly effects the resistance to protease digestion and two others of which are important for permitting export of the protein from the endoplasmic reticulum. Glycosylation-mutants that are altered at these sites (Asn 96, Asn 104, Asn 181, Asn 304, Asn 350, Asn 385) have been prepared using site-directed mutagenesis. These mutations result in the conversion of syngeneic proteins which are normally non-immunogenic into immunogenic altered antigens.

Genetic immunization with a glycosylation mutant syngeneic gp75 where asparagine at amino acid position 350 is altered to delete the glycosylation site at this position was found to stimulate production of autoantibodies against an intracellular, early processed form of gp75. These autoantibodies did not recognize mature gp75.

We have generated these same antibodies by immunizing with cells expressing this altered protein, i.e., immunization with the altered protein has the same effect.

Xenogeneic Differentiation Antigens

In accordance with another embodiment of the present invention, an immune response against a target differentiation antigen can be stimulated by the administration of xenogeneic differentiation antigen of the same type. Thus, for example, an immune response to tumor that expresses gp75 can be stimulated by immunization with gp75 derived from a different species which breaks the tolerance to the autoantigen. For treatments of humans, preferred xenogeneic antigens will be rodent antigens, but could come from other mammals such as dog, cat, cow, or sheep, or from birds, fish, amphibian, reptile, insect or other more distantly related species.

Xenogeneic differentiation antigen may be administered as a purified differentiation antigen derived form the source organism. Proteins can be purified for this purpose from cell lysates using column chromatography procedures. Proteins for this purpose may also be purified from recombinant sources, such as bacterial or yeast clones or mammalian or insect cell lines expressing the desired product. Nucleic acid sequences of various differentiation antigens from various non-human sources are known, including mouse tyrosinase (gp75 (TRP-1)) (Yamamoto et al., Japanese J. Genetics 64: 121-135 (1989)); mouse gp100 (Bailin et al., J. Invest. Dermatol. 106: 24-27 (1996)); and rat prostate-specific membrane antigen (Bzdega et al., J. Neurochem. 69: 2270-2277 (1997). Human TRP-2 (hTRP-2) has 90% homology and 83% identity to the amino acid sequence of mouse TRP-2 (mTRP-2).

Xenogeneic differentiation antigen may also be administered indirectly through genetic immunization of the subject with DNA encoding the differentiation antigen. cDNA encoding the differentiation antigen is combined with a promoter which is effective for expression of the nucleic acid polymer in mammalian cells. This can be accomplished by digesting the nucleic acid polymer with a restriction endonuclease and cloning into a plasmid containing a promoter such as the SV40 promoter, the cytomegalovirus (CMV) promoter or the Rous sarcoma virus (RSV) promoter. The resulting construct is then used as a vaccine for genetic immunization. The nucleic acid polymer could also be cloned into plasmid and viral vectors that are known to transduce mammalian cells. These vectors include retroviral vectors, adenovirus vectors, vaccinia virus vectors, pox virus vectors and adenovirus-associated vectors.

The nucleic acid constructs containing the promoter, antigen-coding region and intracellular sorting region can be administered directly or they can be packaged in liposomes or coated onto colloidal gold particles prior to administration. Techniques for packaging DNA vaccines into liposomes are known in the art, for example from Murray, ed. “Gene Transfer and Expression Protocols” Humana Pres, Clifton, N.J. (1991). Similarly, techniques for coating naked DNA onto gold particles are taught in Yang, “Gene transfer into mammalian somatic cells in vivo”, Crit. Rev. Biotech. 12: 335-356 (1992), and techniques for expression of proteins using viral vectors are found in Adolph, K. ed. “Viral Genome Methods” CRC Press, Florida (1996).

For genetic immunization, the vaccine compositions are preferably administered intradermally, subcutaneously or intramuscularly by injection or by gas driven particle bombardment, and are delivered in an amount effective to stimulate an immune response in the host organism. The compositions may also be administered ex vivo to blood or bone marrow-derived cells (which include APCs) using liposomal transfection, particle bombardment or viral infection (including co-cultivation techniques). The treated cells are then reintroduced back into the subject to be immunized. While it will be understood that the amount of material needed will depend on the immunogenicity of each individual construct and cannot be predicted a priori, the process of determining the appropriate dosage for any given construct is straightforward. Specifically, a series of dosages of increasing size, starting at about 0.1 μg is administered and the resulting immune response is observed, for example by measuring antibody titer using an ELISA assay, detecting CTL response using a chromium release assay or detecting TH (helper T cell) response using a cytokine release assay.

Examples 13-15 demonstrate that active immunization of C57BL/6 mice with xenogeneic DNA coding for human gp100 (hgp100) results in both tumor and autoimmunity. This strategy was able to effect tumor protection to challenge with a murine melanoma known to express the mouse gp100 (mgp100) protein (B16 cells). While immunization of mice with naked DNA coding for hgp100 has been shown by others to be protective against tumor challenge with B16 cells transfected with human gp100, they were not able to demonstrate protective immunity against wild type B16 cells. This important difference may be due in part to the method of DNA immunization.

Naked DNA may lack the advantage of adenovirus transfected dendritic cells which are capable of breaking tolerance to self or helium driven DNA-gold complexes as demonstrated in the following examples. Gold particle bombardment and adenovirus transfected dendritic cells may provide co-stimulation signals that aid in breaking tolerance to mgp100.

Tumor immunity, considered more akin to autoimmunity, has previously been demonstrated to require CD4⁺ help. Similarly, in other animal models of tumor immunity and autoimmunity, CD4⁺ T-cell help is necessary in recognition of a self-molecule. Xenogeneic DNA immunization has been shown to generate antitumor immunity against both gp75^{TRP-1} and TRP-2. Antibodies mediate the immune response to gp₇₅^{TRP-1}, while the immune response to TRP-2 is mediated primarily by T-cells. In both cases tumor rejection required CD4⁺ T-cell help. Example 15 shows that CD4⁺ T-cell help was not required in order to break tolerance to mgp100 and generate effective antitumor immunity. It is possible that other cell types, i.e., CD8⁺ T-cells, could compensate for CD4⁺ T-cell help in MHC II deficient mice, by production of cytokines such as INF-γ. CD4⁺ T-cells have been shown to play important roles in directing immune response, memory and tolerance. It may be possible to improve the antitumor immune response to gp100 by recruiting CD4⁺ T-cells.

As previously shown, immunity against the mgp100₂₅₋₃₃ peptide appears to confer tumor protection. Of the additional MHC class I D^{b} or K^{b} heteroclitic epitopes identified in gp100, none appear to be significantly involved in the response to vaccination with hgp100 DNA by gene gun. In contrast to hTRP-2, the addition of recombinant mouse GM-CSF DNA did not augment antitumor immune response when added to immunization with hgp100 DNA. In addition, GM-CSF DNA did not induce anti-tumor immunity when added to immunization with mgp100 DNA. The benefit of cytokine adjuvants, such as GM-CSF DNA, may depend on the specific antigen and the immunologic cell types involved.

Tumor immunity and autoimmunity are two means to the same end. We have previously observed that vaccination with another xenogeneic DNA against the related gp75^{TRP-1} melanocyte/melanoma differentiation antigen produced the same phenotype in C57BL/6 mice. Immunity against gp75^{TRP-1} led to tumor protection and to depigmentation that was indistinguishable from autoimmunity induced by immunization against TRP-2. Similar results have been observed after immunization with syngeneic gp75^{TRP-1} expressed in vaccinia virus. In gp75^{TRP-1} systems, tumor immunity and autoimmunity were mediated by autoantibodies, without any requirement for CD8⁺ T cells. In striking contrast, tumor immunity and autoimmunity after xenogeneic immunization against TRP-2 required MHC class I molecules and by implication CD8⁺ T cells, without a requirement for antibodies. Thus either autoantibodies or cytotoxic T cells can provide specificity that mediates tumor immunity and autoimmunity. In the end, the phenotypes produced by antibody and T cell mediated immunity in these models are hard to distinguish at the level of the host, since both lead to tumor rejection and depigmentation.

Active immunization with xenogeneic TRP-2 induced more rapid tumor immunity and autoimmunity than immunization with xenogeneic gp75^{TRP-1}. Vaccination against TRP-2 as late as 10 days after tumor challenge induced substantial decreases in tumor burden while active immunization against gp75^{TRP-1} at these later time points was ineffective. We propose this reflects the kinetics of T cell versus antibody responses, where effective T cell immunity can be generated over days while effective antibodies may require weeks. It is also possible that antibodies are more effective during the blood-born stage of disease while T cells are most effective for tumors in tissues. CD4⁺ T cells were required for both antibody-mediated and cytotoxic T cell-mediated immunity, emphasizing how central CD4⁺ T cells are for tumor immunity.

The potential coupling of tumor immunity with autoimmunity has been suggested by the clinical observation that patients with metastatic melanoma who develop depigmentation may have a better prognosis and are more likely to have responded to therapy. The differences in mechanisms underlying tumor immunity and autoimmunity could be a consequence of fundamental differences in effector mechanisms used to kill tumor cells versus normal melanocytes. Alternatively the differences could reflect the different tissue sites of melanocytic cells. In this scenario, the effector mechanisms in the skin require perforin while other mechanisms are used in the lung. Our present studies are investigating the potential role of organ and tissue sites. Mechanisms other than perforin or fas ligand may be involved in tumor rejection, but it is also possible that perforin and fas ligand provide redundant mechanisms for effector functions against tumors. Finally, the uncoupling of tumor immunity from autoimmunity in this model shows that one can block autoimmunity and still be permissive for tumor immunity. This opens strategies for inducing immunity to treat cancer where autoimmunity can be inhibited while cancer immunity proceeds.

The invention will now be further described with reference to the following, non-limiting examples.

### EXAMPLE 1

C57BL/6 mice (National Cancer Institute, Bethesda, Md.) were immunized with (a) syngeneic gp75⁺ B16 melanoma cells (which express a non-mutant b locus protein); (b) syngeneic B16 cells expressing IL-2, GM-CSF and IFN-γ; (c) syngeneic gp75⁻ B16 melanoma variant, B78H.1 and syngeneic fibroblasts transfected with cDNA expressing the mouse b allele; (d) hydrophilic peptides of gp75 conjugated to carrier protein; and (e) full length gp75 glycoprotein purified from syngeneic melanoma cells. Cells, purified glycoprotein or peptides were combined with adjuvants, including Freund's adjuvant, a mixture of bacterial cell wall skeletons and an endotoxin derivative (DETOX), and a saponin component (QS21). Immunizations were performed by intraperitoneal, subcutaneous and intradermal routes. After immunizations, mice were assessed for antibodies against gp75 by ELISA, immunoprecipitation and Western blots, and for cytotoxic T lymphocytes (CTL) to B16 using a ⁵¹Cr-release cell-mediated cytotoxicity assay. As summarized in Table 1, no antibodies or CTL against gp75 were detected after any of these immunization strategies, supporting the conclusion that C57BL/6 maintain tolerance to the gp75 glycoprotein.

TABLE 1

| Source of gp75 | Adjuv | # Inj | Route | Dose | Ab/CTL Response |
|---|---|---|---|---|---|
| B16 melanoma | none | 1 | sc, iv | 50,00 | no response |
| Irradiated B16 | none | 5 | ip | 8 × 10⁶ | no response |
| Irradiated B16 | F, D | 5 | ip | 8 × 10⁶ | no response |
| 3.27 tx | none | 5 | ip, sc | 8 × 10⁶ | no response |
| 3.27 tx | F, D | 5 | ip, sc | 8 × 10⁶ | no response |
| TIB88/gp75 tx | F | 4 | ip, sc | 5 × 10⁶ | no response |
| B16-IFNy | none | 5 | sc, iv | 8 × 10⁶ | no response |
| B16-IL-2 | none | 5 | sc, iv | 8 × 10⁶ | no response |
| B16-IL-2/IFNy | none | 5 | sc, iv | 8 × 10⁶ | no response |
| B16-GM-CSF | none | 5 | sc, iv | 8 × 10⁶ | no response |
| Purified gp75 | | 5 | sc, iv | 10 μg | no response |
| gp75 peptides | F | 5 | id | 100 | no response |

Dose refers to individual dose per injection, either number of cells per injection or amount of protein or peptide. One week following the last immunization, sera were tested by immunoprecipitation and Western blots against lysates of syngeneic B16F10 melanoma and ELISA for antibodies (Ab) to gp75 and splenocytes were tested for CTL responses. Adjuv=adjuvant, F=Freund's adjuvant, D=Detox adjuvant, Q=QS21 adjuvant, #inj=number of immunizations at 7-14 day intervals. Route of immunization: sc=subcutaneous, iv=intravenous, id=intradermal, B16-IFNγ, B16-IL-2 and B16-GM-CSF CSF refer to B16F16 melanoma cells expressing the designated cytokine. Tx=transfectant. 3.27 are B78 H.1 melanoma cells transfected with gp75, and TlB88/gp75 are syngeneic fibroblasts transfected with gp75.

### EXAMPLE 2

As in Example 1, syngeneic C57BL/6 mice immunized with either cell-associated or purified forms of gp75 protein did not produce autoantibodies to gp75. We next assessed whether gp75 encoded by cDNA delivered into the dermis of syngeneic C57BL/6 mice by particle bombardment would induce an autoantibody response.

C57BL/6 mice were genetically immunized with cDNA encoding full-length syngeneic gp75 under the control of a CMV promoter once a week for five weeks. Sera from these mice were then assessed for autoantibodies against gp75 by immunoprecipitation. No mouse (0 out of 28) had detectable antibodies, indicating that C57BL/6 mice maintained their tolerance to the syngeneic protein.

### EXAMPLE 3

A baculovirus expression vector encoding full length murine gp75 was constructed and isolated in collaboration with Dr. Charles Tackney (Imclone, New York, N.Y.) using standard techniques. Summers & Smith, “A manual for methods for baculovirus vectors and insect cell culture procedures”, Texas Agricultural Experiment Station Bulletin No 1555 (1987); Lucklow & Summers, Biotechnology 6:47-55 (1988). Briefly, the 1.8 kb EcoRI fragment of pHOMERB2 encoding murine gp75 was subdloned into a baculovirus expression vector related to pBbac produced by Stratagene, Inc, and the expression vector introduced into baculovirus. Spodoptera frugiperda Sf9 insect cells were co-infected with this virus construct and wild-type Autographa californica nuclear polyhedrosis virus (AcNPV) and recombinant AcNPV expressing mouse gp75 was generated by homologous recombination. After plaque purification, Sf9 cells were infected with the recombinant virus and clones expressing high levels of gp75 were identified by screening with an antibody against gp75. These cell lines were used for immunization studies.

C57BL/6 mice were immunized with lysates of insect Sf9 cells expressing either syngeneic gp75 in a baculovirus vector (gp75/Sf9) or wild-type baculovirus (wt/Sf9). Mice immunized with gp75/Sf9 lysates (1 or 5×10⁶ cells) developed autoantibodies to gp75 with (120/120 mice) or without (25/28 mice) Freund's adjuvant. No antibodies were detected after immunization with wt/Sf9 (0 of 46 mice). Autoantibodies appeared after two to four immunizations, lasted for more than four months after the last immunization, and reacted with gp75 expressed in syngeneic melanocytic cells (B16F10 and JBRH melanomas). B16F10/LM3 (B16) is a pigmented mouse melanoma cell line of C57BL/6 origin derived from the B16F10 line provided by Dr. Isaiah Fidler (M. D. Anderson Cancer Center, Houston, Tex.). Antibodies were IgG class, based on reactivity with rabbit anti-mouse IgG and protein A, and copurification of antibody reactivity with IgG fractions from sera.

The difference in immunogenicity between gp75/Sf9 and mouse gp75 was not due simply to quantitative differences in the amount of gp75 in the two preparations. 8×10⁶ B16 melanoma cells contained 20 μg of gp75, compared to only 14 μg in 1×10⁶ gp75/Sf9 cells. Also, 10 μg of purified mouse gp75 mixed with wt/Sf9 lysates did not induce autoantibodies. Although Sf9 cells can apparently provide an adjuvant effect (Prehaud et al., Virology 173: 390-399 (1989); Ghiasi et al., J. Gen. Virology 73: 719-722 (1992)), these results show that other differences between gp75 produced in mouse cells versus insect cells (for instance carbohydrate structures) were necessary to induce autoantibodies.

### EXAMPLE 4

In contrast to immunization with gp75/Sf9 lysates, immunization with purified gp75 (12 μg) produced in gp75/Sf9 insect cells plus Freund's adjuvant induced autoantibodies that recognized 683/70 kDa early processed forms of gp75. This form of gp75 contained only immature, high mannose-linked carbohydrates, which results in localization of the molecule to the endoplasmic reticulum or cis Golgi compartment.

### EXAMPLE 5

Mice were immunized with the gp75⁺ human melanoma cell line SK-MEL-19 with Freund's adjuvant and evaluated for the development of autoantibodies to murine gp75. All of the mice (20/20) developed autoantibodies. There was no response without adjuvant (0/5 mice), and no antibodies to gp75 were detected in sera of 12 mice immunized with gp75⁻ human melanomas SK-MEL-131 or SK-MEL-37 plus Freund's adjuvant. Three of five mice immunized with purified human gp75 (10 μg per dose for five immunizations) with Freund's adjuvant developed autoantibodies to gp75, although the antibody responses were generally weaker, possibly due to the lower amount of purified gp75 used compared to the amount of gp75 in SK-MEL-19 lysates. Thus, administration of human gp75 broke the tolerance to gp75 in C57BL/6 mice.

### EXAMPLE 6

B16 melanoma cells and normal melanocytes in C57BL/6 mice express gp75, the wild-type b allele of the brown locus. As described above, the product of this locus is recognized by sera from syngeneic mice immunized with mouse gp75 expressed in gp75/Sf9 cells and human gp75. We have previously shown that passive transfer of mouse monoclonal antibody against gp75 into mice bearing B16F10 tumors leads to tumor rejection. Hara et al., Int. J. Cancer 61: 253-260 (1995). To determine whether the autoimmune responses observed conferred similar protection against tumors, the in vivo effects of immune recognition of gp75 were investigated using a syngeneic tumor model.

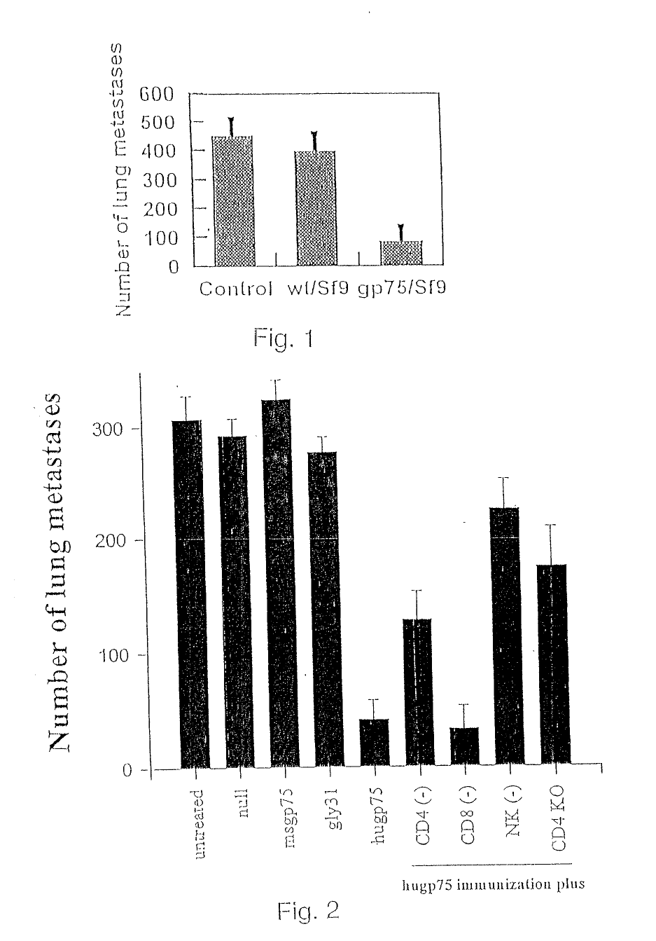

Mice (5 mice per group) were injected subcutaneously with gp75/Sf9 lysates (5×10⁶ gp75/Sf9 cells) concurrently with 10⁵ B16F10 melanoma cells administered intravenously and the occurrence of lung metastases 14 days after tumor challenge was monitored. Mice immunized with wt/Sf9 cells and unimmunized mice were used as controls. The results are summarized in FIG. 1. As shown, mice immunized with gp75/Sf9 lysates were substantially protected against formation of lung metastases compared to the controls. Significant protection (53% decrease in lung metastases) was also observed when immunization was carried out 4 days after the tumor challenge as metastases become established. There was no significant protection in mice immunized with wt/Sf9 lysates compared to the unimmunized control.

Passive transfer of serum from mice immunized with gp75/Sf9 to five unimmunized mice produced a 63% decrease in lung metastases compared to mice treated with an equivalent amount of normal mouse serum (p=0.02), supporting the conclusion that tumor protection was at least partially mediated by humoral mechanisms.

Mice immunized with human gp75⁺ SK-MEL-19 were also markedly protected against B16F10 melanoma compared to unimmunized mice. (4+/−7 metastases in immunized mice versus 275+/−77 lung metastases in control mice—6 mice per group). Immunization with gp75⁻melanoma SK-MEL-131 did not introduce tumor protection against B16F10 melanoma, although recognition of other xenogeneic antigens other than gp75 could not be critically assessed.

Mice immunized against the immature, early processed form of gp75, using purified gp75 from gp75/Sf9 cells were not significantly protected against B16F10 metastases (366+/−78 metastases in four immunized mice versus 412+/−94 metastases in five unimmunized control mice). However one mouse in this group did eventually develop autoantibodies against mature gp75 and was protected against lung metastases (only 21 metastases).

### EXAMPLE 7

C57BL/6 mice were genetically immunized with cDNA encoding full length human gp75 under control of the control of a CMV promoter once a week for five weeks by gene gun injection. As controls, mice were injected with full length syngeneic mouse gp75 under the control of the CMV promoter, with a glycosylation mutant of gp75 (gly31) or null DNA. Four weeks after the final immunization, the mice were injected through the tail vein with 2×10⁵ B16F10LM3 melanoma cells. One group of treated mice were also challenged with melanoma cells. Twenty-one days after tumor challenge, mice were sacrificed and surface metastatic lung nodules were scored. There were ten mice in the untreated group, 9 mice in each of the null and mouse gp75 groups, 8 mice in the gly31 group and 19 mice in the human gp75 group. The importance of CD4⁺, CD8⁺ and NK cells was also tested by depletion using monoclonal antibodies (rat mAb GK1.5 for CD4; mAb 53.6.7 for CD8 and mAb PK1.36 for NK1.1). The necessity of CD4⁺ T cells was also assessed by looking for tumor rejection in CD4 knock-out mice (CD4 KO) after in vivo transfer of the human gp75 gene by gene gun.

As shown in FIG. 2, mice immunized with xenogeneic human gp75 were found to be significantly protected from lung metastases (mean 41±15 metastases) when challenged with B16F10LM3 melanoma (p<0.0001), with an 84% decrease in lung nodules as compared with control mice. Syngeneic mice that received in vivo gene transfer of the glycosylation mutant mouse gp75 were not significantly protected from B16F10LM3 tumor challenge (mean 300±12 metastases), nor were those that were delivered control DNA (mean 29:2±15 metastases) by particle bombardment or were left untreated (mean 307±20 metastases) (p>0.45). CD8⁺ deletion did not alter tumor rejection, although depletion of CD4⁺ (by mAb or knock-out) and NK1.1⁺ cells did result in a reduction in level of protection achieved. Thus these latter cells may play a role in the protection against tumors achieved using genetic immunization with xenogeneic DNA.

### EXAMPLE 8

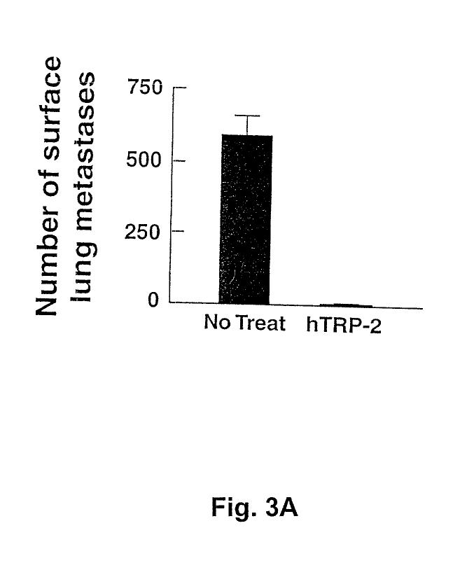

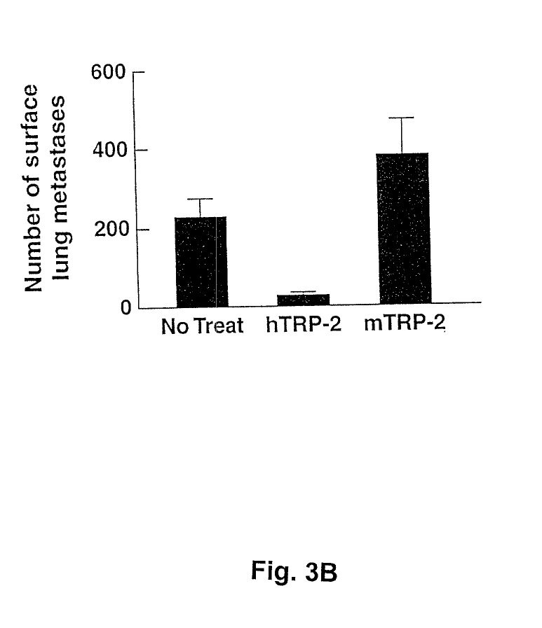

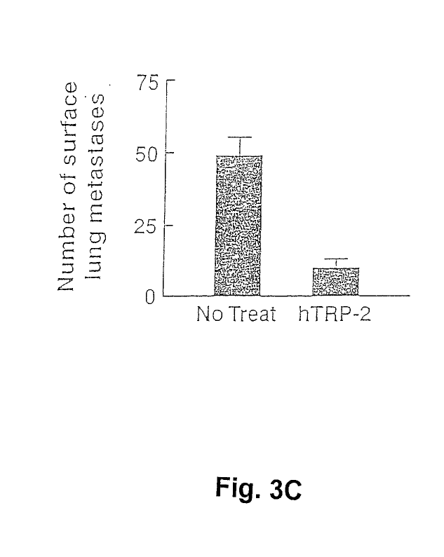

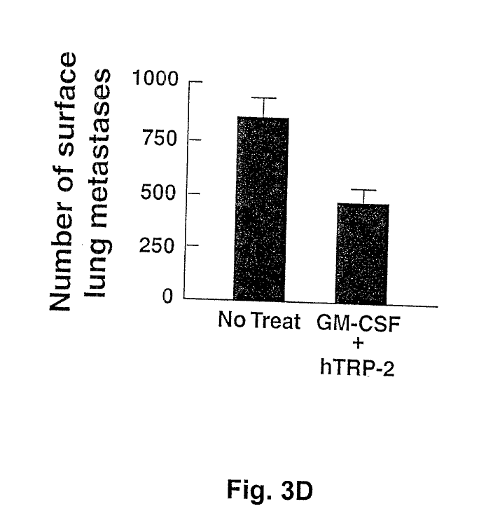

Xenogeneic but not syngeneic TRP-2 DNA induces tumor rejection including established tumors. C57BL/6 mice, 10-12 per group, were immunized cutaneously by DNA immunization using particle bombardment with gold particles coated with plasmid DNA encoding syngeneic mTRP-2 or the xenogeneic hTRP-2 gene. Mice were challenged intravenously with B16BF10/LM3 melanoma cells and lung metastases scored after approximately three weeks. Mice were immunized three times at weekly intervals wtih hTRP-2 and compared to untreated mice. Mice were challenged with B16F10/LM3 melanoma cells 5 days after the last immunization. DNA immunization with xenogeneic hTRP-2 decreased lung metastases by ≧95% (p<0.0001) in tumor protection experiments (FIG. 3A). Mice were immunized five times at weekly intervals with hTRP-2, mTRP-2 or were untreated, and were challenged with B16F10/LM3 melanoma cells 5 days after the last immunization. Significant protection was observed with hTRP-2 compared to no treatment (p=0.001) or to mTRP-2 (p=0.001). The difference between mice treated with mTRP-2 and untreated mice was not significant (p=0.16), although there was a trend for increased tumors (FIG. 3B). Differences in protein sequence between human and mouse TRP-2 may have been sufficient to generate immunity. Initial recognition of hTRP-2 triggered immunity against self TRP-2 in the mouse.

To assess the potency of DNA immunization using xenogeneic hTRP-2 DNA, mice were immunized four days after tumor challenge when lung metastases were established or 10 days after tumor challenge when lung metastases were numerous and macroscopic. Immunization at four days decreased metastases by >80% (p<0.001) (FIG. 3C). Therapeutic: effects were observed 10 days after tumor challenge using immunization with hTRP-2 DNA plus recombinant mouse GM-CSF DNA as an immune adjuvant. Vaccination significantly decreased lung metastases by ˜half (p=0.004) (FIG. 3D). No significant decrease in lung metastases was observed after treatment with hTRP-2 (783±75 nmetastases) or mTRP-2 DNA (902±65 metastases) or GM-CSF DNA alone (692±69 metastases) for 10 day tumors (null control vector gave 705±61 metastases), showing a requirement for xenogeneic antigen and the immune adjuvant effect of GM-CSF in treatment of rapidly growing established tumors.

### EXAMPLE 9

Xenogeneic hTRP-2 DNA vaccination induces autoantibodies and autoreactive cytotoxic T cells. Next, it was determined whether immunization with mTRP-2 or xenogeneic hTRP-2 generated antibody and CD8⁺ T cell responses against syngeneic mouse TRP-2. Groups of 10-13 mice were immunized with DNA encoding mouse TRP-2 (mTRP-2) or human TRP-2 (hTRP-2) five times. Serum from each mouse was tested for antibodies against mTRP-2 by immunoprecipitation-Western blot assays as described by Weber, L. W., et al. J. Clin. Invest. 102:1258-64 (1998). B 16F10/LM3 melanoma cells were lysed, followed by immunoprecipitation with PEP-8, a rabbit polyclonal antisera raised against a carboxyl-terminal peptide of TRP-2 (Vincent Hearing, NCI). Immunoprecipitates were separated by 8% SDS-PAGE under reducing conditions and transferred to Imnnobilon membranes (Millipore, Bedford, Mass.). The membranes were probed with mouse sera or preimmune mouse sera at 1:60 dilution.

Approximately half of the mice immunized with hTRP-2 had detectable IgG antibodies (IgG1 and IgG2b isotype) against mTRP-2 in sera (Table 2).

TABLE 2

| | | Antibody |
|---|---|---|
| Immunized Mouse Strain | DNA Immunization | Responders/Total |
| C57BL/6 wt | mTRP-2 | 0/12 |
| C57BL/6 wt | hTRP-2 | 6/12 |
| β2m−/− | hTRP-2 | 0/11 |
| MHC II−/− | hTRP-2 | 0/10 |
| NK depleted | hTRP-2 | 3/13 |
| μ−/− | hTRP-2 | 0/12 |

No autoantibodies against syngeneic mouse TRP-2 were generated after immunization with mTRP-2. Generation of autoantibodies after immunization with human TRP-2 required both CD4⁺ and CD8⁺ T cells because no autoantibodies were detected in mice deficient in MHC class I (β2m−/−) or class II molecules (MHC II −/−). Mice with homozygous genetic deficiencies; for β2-microglobulin (β2m-−/−) and MHC II (Abb-−/−) on a C57BL/6 background were obtained from Taconic Farms, Inc. (White Plains, N.Y.). Immunoglobulin μ chain (Igμ-I-) mice were acquired from The Jackson Laboratory (Bar Harbor, Me.).

Cytotoxic T cell (CTL) responses against TRP-2 were detected after immunization with xenogeneic hTRP-2 (FIG. 4C) but not syngeneic mTRP-2 (FIG. 4B) DNA, as compared to non-immunized (FIG. 4A). C57BL/6 mice (2-3/group) were immunized as described above. Seven days after the last immunization, draining lymph nodes were pooled and stimulated for five days and tested for cytotoxicity against EL-4 target cells, either pulsed with TRP-2 peptide or unpulsed. The EL-4 cell line was derived from a C57BL/6 mouse thymoma. Briefly, lymphocytes (2×10⁷) from draining inguinal lymph nodes were co-cultured for 5 days with na,ve irradiated (3,000 rads) splenocytes (2×10⁷), mitomycin C (100 μg/ml, 120 minutes at 37° C.) treated human SK-MEL-188 ells (4×10⁵) as a source of antigen, and pristane-induced macrophages (2×10⁶) from C57BL/6 mice. H-2K^{b} 0restricted lymphocyte lysis of EL-4 tumor cells pulsed for 1 hour at 37° C. with 4 μg mTRP-2₁₈₁₋₁₈₈ peptide (sequence VYDFFVWL) was determined by ⁵¹Cr release assay. A standard ⁵¹Cr release assay was used to measure cytotoxicity. Naftzger, C., et al., , Proc. Natl. Acad. Sci. USA 93:14809-14814 (1996).

Specifically, CD8⁺ CTL from draining lymph nodes recognized an MHC class I H-2K^{b} restricted peptide of mTRP-2 after immunization with hTRP-2 DNA (FIG. 4C), and specifically killed B16 melanoma cells. Interestingly the H-2k^{b} restricted peptide of mTRP-2, mTRP-2₁₈₁₋₁₈₈, is identical between mouse and human TRP-2 including flanking amino acid residues. Thus, presentation of this self peptide in the context of self TRP-2 DNA does not induce CTL responses, but presentation of the same peptide in the context of xenogeneic hTRP-2 is immunogenic. Amino acid differences in other peptides of hTRP-2 may provide T cell help, which in turn is sufficient to trigger cytotoxic T cell responses to the mTRP-2₁₈₁₋₁₈₈ self peptide. The observation that only hTRP-2 DNA (but not mTRP-2 DNA) induced CD8⁺ T cell responses suggests that T cell tolerance was broken by xenogeneic DNA immunization.

### EXAMPLE 10

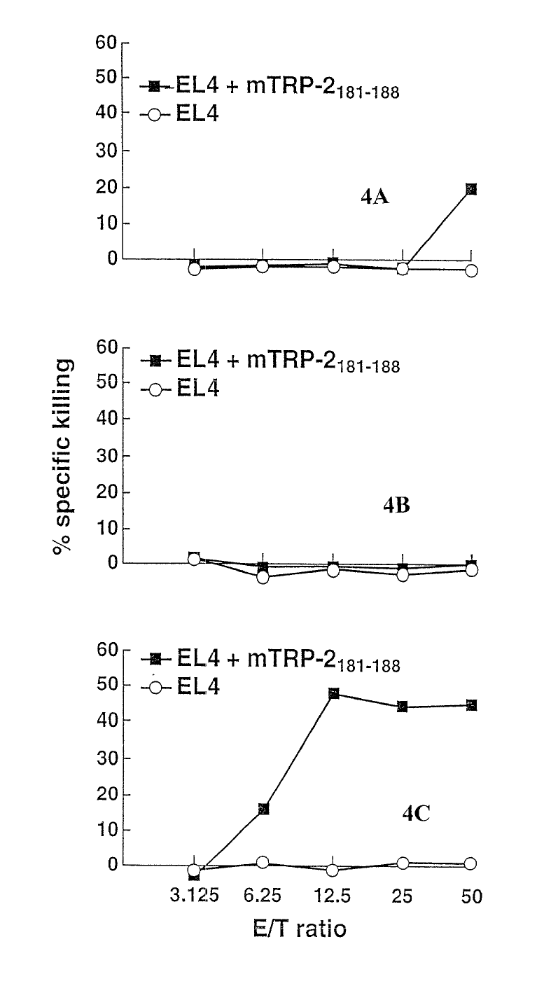

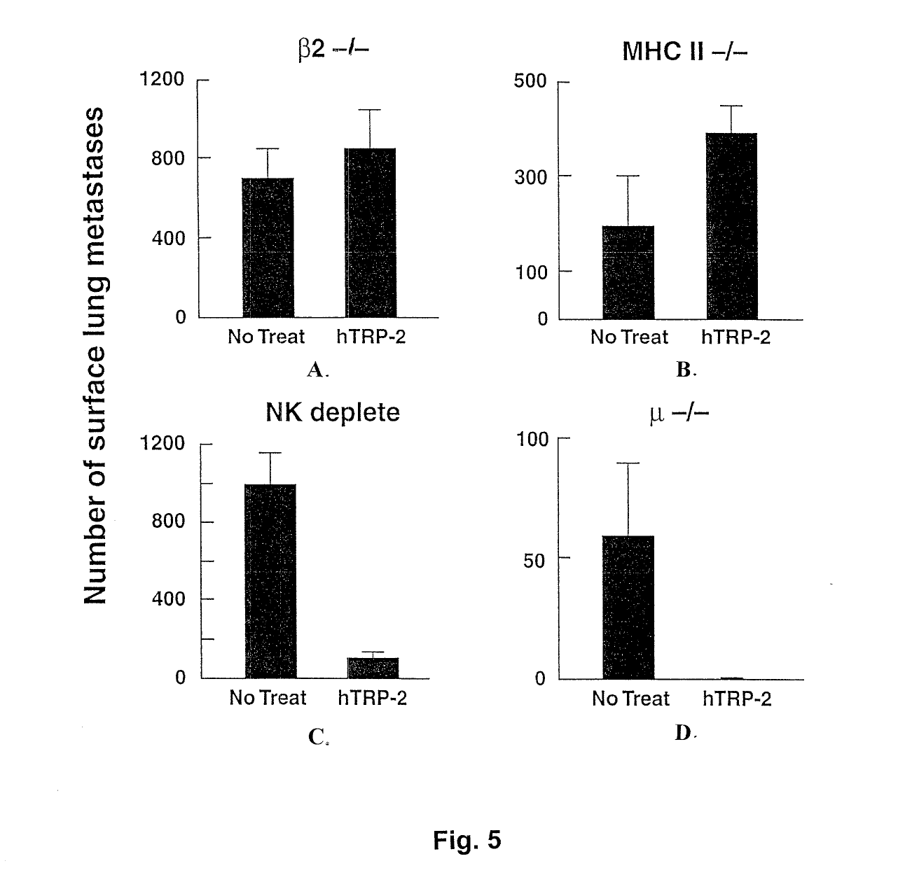

Tumor rejection requires CD4⁺ and CD8⁺ T cells but not B cells or natural killer cells. These results suggested that either antibody or cytotoxic T cell responses, or both, mediated tumor rejection. Roles for critical cell types were investigated by immunizing β2m−/− mice deficient in MHC class I (and CD8⁺ T cells) (FIG. 5A), MHC II −/− mice deficient in MHC class II (and CD4⁺ T cells) (FIG. 5B), mice depleted of NK1.1⁺ cells (FIG. 5C), and Igμ−/mice deficient in B cells (FIG. 5D) with hTRP-2 DNA. Significant protection was observed in NK depleted (p<0.0001) and μ−/−/− (p=0.03) but not μ2−/− or MHC II −/− (p>0.10) mice. T cell immunity was required for tumor rejection, supporting a central role for both CD8⁺ and CD4⁺ T cells. Neither NK cells nor B cells were necessary for tumor immunity. Noticeably mice deficient in B cells developed fewer metastases and were completely free of any detectable tumor after treatment with human TRP-2 (12 of 12 mice). This phenomenon of enhanced T cell-dependent tumor rejection associated with B cells deficiency has been reported previously. These results showed that T cell immunity, including both CD8⁺ and CD4⁺ T cells, was required for tumor rejection, but antibodies were not.

### EXAMPLE 11

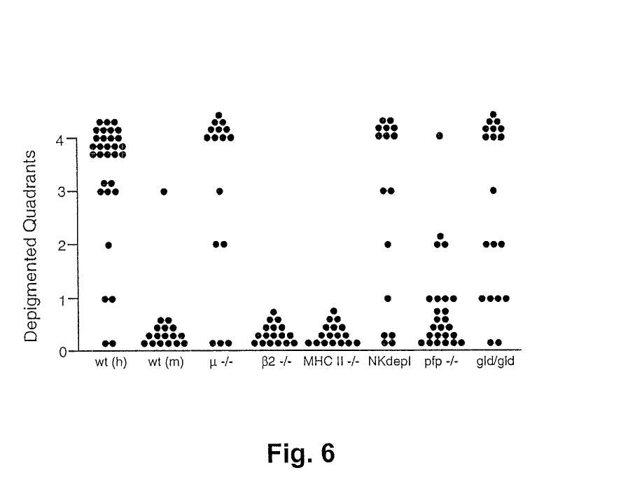

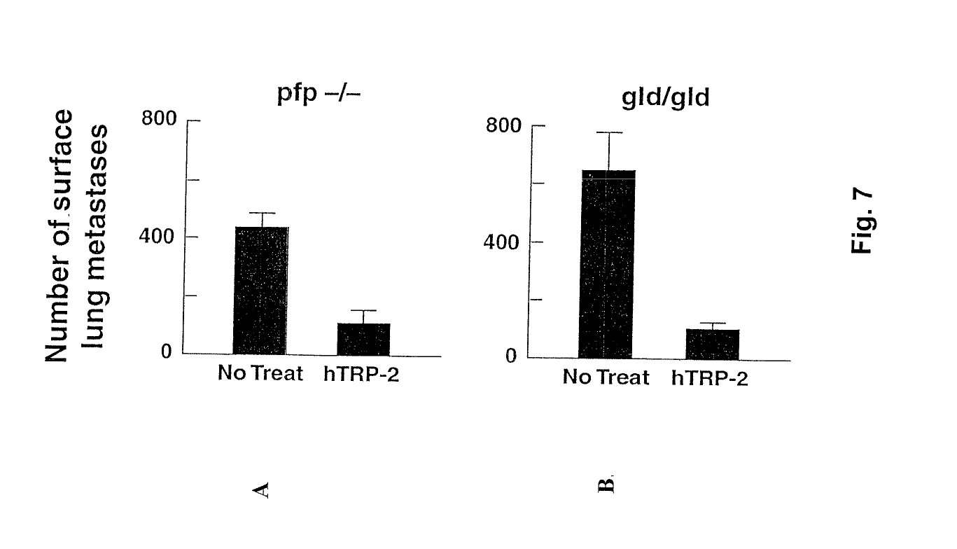

Xenogeneic immunization induces autoimmunity that also requires T cells. Signs of autoimmunity, manifested as depigmentation, were observed in mice immunized with human TRP-2 but not in mice immunized with syngeneic mouse TRP-2 (with one exception) (FIGS. 6 and 7). Following the final immunization mice were shaved and depilated over the posterior flank and observed for coat color changes upon regrowth of hair, following mice for six to eight weeks. Scoring of depigmentation was preformed by dividing the abdomen into four equal quadrants. Quadrants were scored and recorded as positive when they had >50% depigmented hairs. Depigmentation was scored 0-4+ according to the number of quadrants that were depigmented in each mouse with 1+ added for each depigmented quadrant (e.g., 3 if three of four quadrants are depigmented >50%). Depigmentation first appeared 4-5 weeks after starting DNA immunization, and was most prominent over depilated and shaved areas of the mouse coat. Depigmentation spread to unshaved areas in most mice. Autoimmunity also required T cells but not antibodies or NK cells, showing that tumor immunity and autoimmunity were coupled by a requirement for class I and class II MHC expression leading to a requirement for T cells.

### EXAMPLE 12

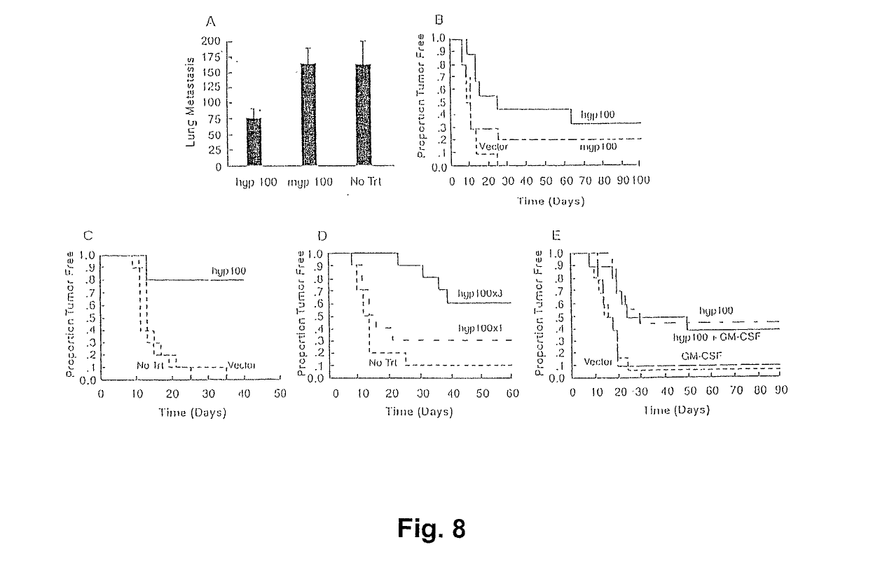

Perforin is required in T cell dependent autoimmunity but not tumor immunity. In many if not most mouse transplantable tumor models, cytotoxic T cells have been proposed to be the critical effector cells that mediate tumor rejection. Cytotoxicity of T cells can be mediated by exocytic, granules involving perforin or by cell membrane molecules, including fas ligand and TRAIL, that induce death of target cells. Despite the requirement for T cells in both tumor immunity and autoimmunity, these two events were uncoupled at the effector stage. Tumor immunity proceeded in the complete absence of perforin (P−0.0002) in pfp−/− mice (FIG. 6) while autoimmunity was largely abrogated (FIG. 7). Fas ligand was not necessary for either autoimmunity (FIG. 7) or tumor immunity (FIGS. 8A and 8B). These results are consistent with perforin-mediated killing of normal melanocytes in hair follicles playing a central role in autoimmunity. However, tumor immunity could proceed in a perforin-independent manner.

Tissue-specific molecules have emerged as a major set of autoantigens recognized by the immune system of patients with melanoma. TRP-2 is recognized by cytotoxic T cells of patients with melanoma and has also been defined as a potential tumor rejection antigen in C57BL(6 mice. Thus TRP-2 provides an excellent model for a differentiation antigen with relevance to a human cancer. Xenogeneic DNA vaccination is one strategy to immunize against potentially weak self antigens.

### EXAMPLE 13

Tumor protection was assessed in two systems: intravenous and intradermal tumor challenge. The intravenous route was used to model metastatic disease, while intradermal tumor challenge was used to model local growth of melanoma in the skin. For the lung metastasis experiments, mice were injected intravenously via tail vein with 2×10 B16 melanoma cells five days after the final immunization with human gp100 DNA. Mice were sacrificed 14 days after tumor challenge, all lobes of both lungs were dissected, and surface lung metastases were counted under a dissecting microscope. Statistical analysis was performed using the two-sided Student's t test. For the intradermal tumor experiments, mice were injected intradermally with 1×10 B16 melanoma cells on the right flank five days after the final immunization. The mice were palpated for the presence of tumors, and average tumor diameter was measured with calipers every other day. Tumors were scored as present once they reached an average diameter of two mm. Mice were sacrificed if tumors reached an average diameter of two cm or if they ulcerated. Kaplan-Meier tumor free survival curves were constructed and Log Rank statistical analysis was performed to determine significance.

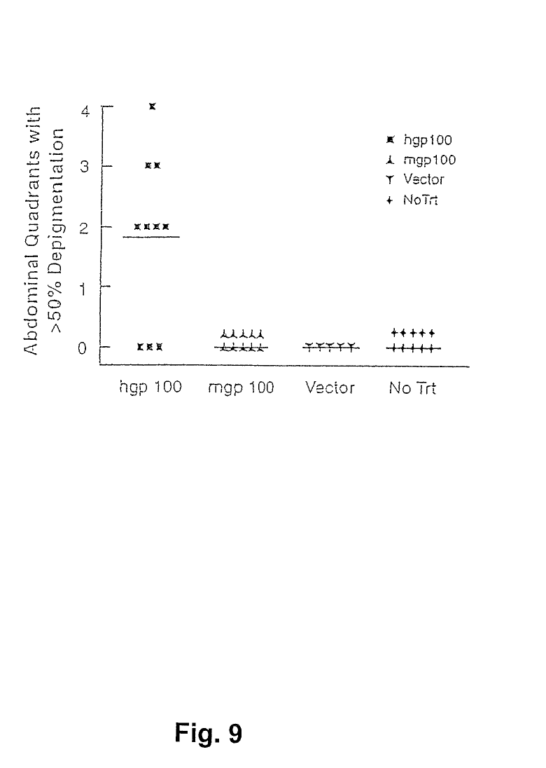

DNA immunization of mice with xenogeneic hgp100 decreased lung metastasis by 50% (p <0.03) in mice challenged intravenously with B16 melanoma (FIG. 9A). Following an intradermal tumor challenge long-term tumor-free survival was noted in mice immunized with hgp100 DNA. In these mice 30 and 90 day tumor=free survival was noted in 27 of 40 mice (68%) (4 experiments), and 14 of 30 mice (37%) (3 experiments) respectively (FIGS. 9B-9E). Only 2 of 50 mice (4%) in the untreated and/or vector alone groups were tumor free at 30 days (4 experiements). Three weekly immunizations resulted in a statistically significant improvement in tumor-free survival compared to a single immunization (p <0.05) or no treatment (FIG. 9D) (p <0.001). Recombinant mouse GM-CSF DNA was tested as an immune adjuvant in combination with hgp 100 but did not provide any additional antitumor effect (FIG. 9E).

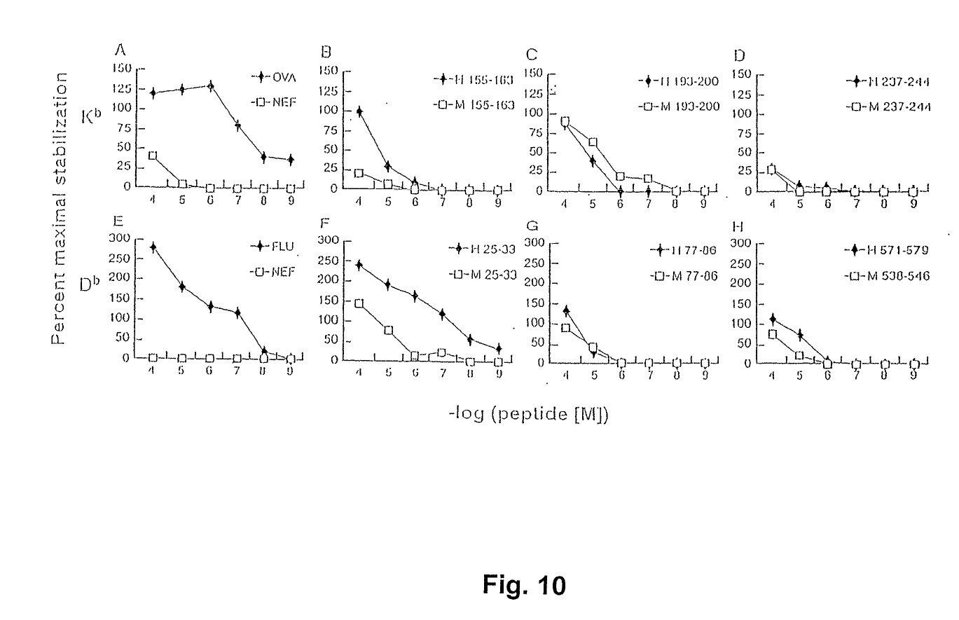

The induction of autoimmunity against melanocytes caused by vaccination with melanosomal antigens manifests as coat color depigmentation that can be measured by degree of severity. For the depigmentation experiments, after the final immunization, hair was allowed to regrow over the abdomen after having been shaved and depilated for immunizations. At six weeks after the final immunization, scoring for depigmentation was performed by dividing the abdomen into four equal quadrants. Quadrants were recorded as positive when >50% of hairs were depigmented. Scores from zero to four were generated for each mouse according to the number of quadrants that were depigmented. Chi squared tables were constructed for presence or absence of depigmentation by group, and data analyzed by Fisher's exact test. Depigmentation was observed in 7 of 10 mice immunized with hgp 100 but not in any mice immunized with syngeneic mgp100, empty vector, or untreated mice (FIG. 10). Depigmentation appeared six weeks after the first immunization in depilated and shaved areas of the mouse coat, occasionally spreading to unshaved areas by six months.

### EXAMPLE 14

Xenogeneic gp100 DNA vaccination results in auto-reactive cytotoxic T-cells and sporadic low-titer autoantibodies. It was next determined whether immunization with hgp100 or mgp100 resulted in CD8⁺ T-cell or antibody responses against the syngeneic mgp100. An MHC class I epitope restricted by D^{b} in mgp100 has previously been published. Overwijk, W. W., et, al., J. Exp. Med. 188(2):277-286 (1998). T-cells stimulated ex vivo with this peptide exerted a tumor protective effect when adoptively transferred into naive mice challenged with B16. We sought to determine if CD8⁺ T-cells from mice immunized with hgp100 reacted with this epitope as well as to assess the importance of other possible epitopes.

The amino acid sequences of mouse and human gp100 were screened for potential heteroclitic differences based on known structural binding motifs of the K^{b} and D^{b} MHC class I pockets. Heteroclitic peptides are variant peptides of higher biologic potency than the wild type peptide that can be attributed to increased binding to the MHC class I molecule. Six pairs of potential heteroclitic peptides including the previously published hgp100₂₅₋₃₃ were identified and are listed in Table 3.

TABLE 3
Heteroclitic differences

| MHC I | Human/Mouse | Position |
|---|---|---|
| K^{b} | TWGQYWQVL | 155-163 |
| | TWGKYWQVL | 155-163 |
| K^{b} | GSRSYVPL | 193-200 |
| | GSQSYVPL | 193-200 |
| K^{b} | PLTFALQL | 237-244 |
| | PLIFALQL | 237-244 |
| D^{b} | KVPRNQDWL | 25-33 |
| | EGSRNQDWL | 25-33 |
| D^{b} | LIGANASFI | 77-86 |
| | LVGANASFI | 77-86 |
| D^{b} | LADTNSLAV | 571-579 |
| | LADANSLAV | 538-546 |

Underlines indicate anchor residues for binding to K^{b} or D^{b}.

Peptide-MHC binding was quantified by determining relative expression of stable MHC molecules on the transporter antigen peptide (TAP)-deficient cell line RMA-S in the presence of indicated peptides. All peptides listed in Table 3 and controls were obtained from Research Genetics Inc. (Huntsville, Ala.). Percent maximal stabilization was calculated relative to the levels of total (empty and peptide-associated) MHC class I molecules at 29° C. as (MFI with peptide at 37° C.−MFI without peptide at 37° C.)/(MFI at 29° C.−MFI without peptide at 370° C.)×100, where MFI=mean fluorescence intensity. Ovalbumin (OVA) peptide (SIINFIEKL) served as a positive binding control for K^{b} and Influenza (FLU) peptide (ASNENMETM) served as a positive control for D^{b}. HIV-NEF peptide (VLEWRFDSRL) served as a negative control for both K^{b} and D^{b}.

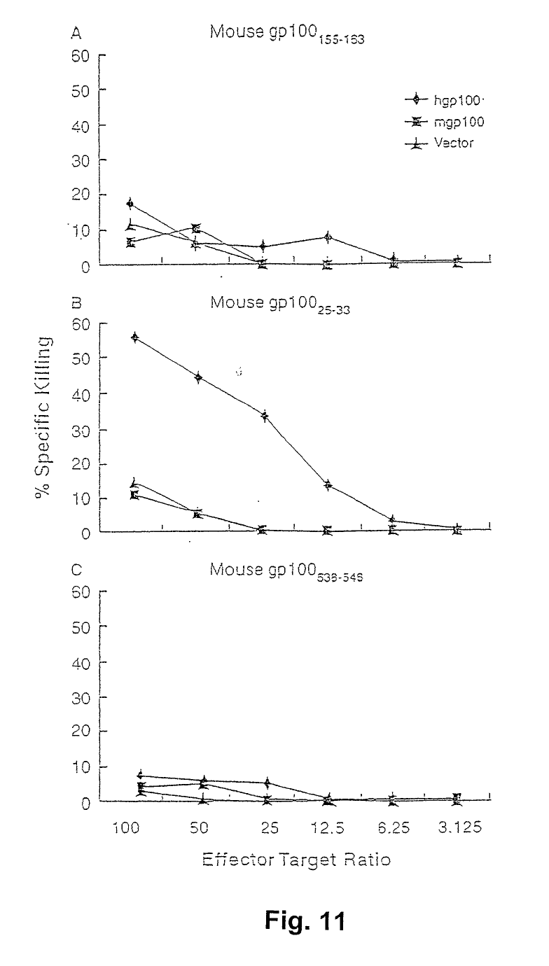

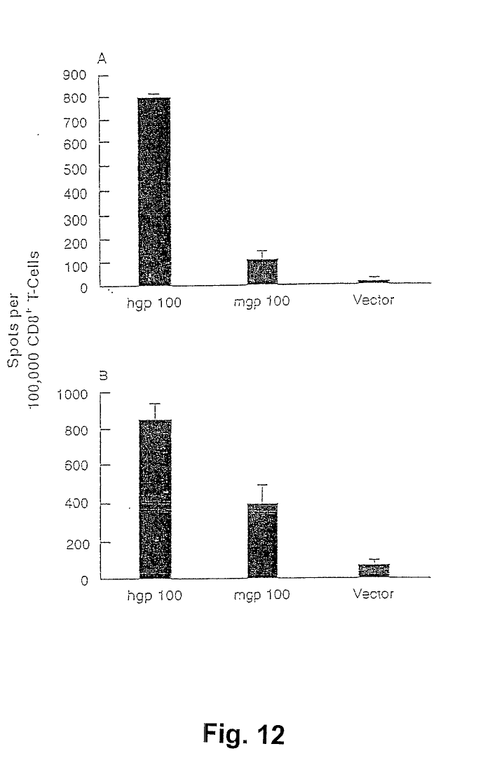

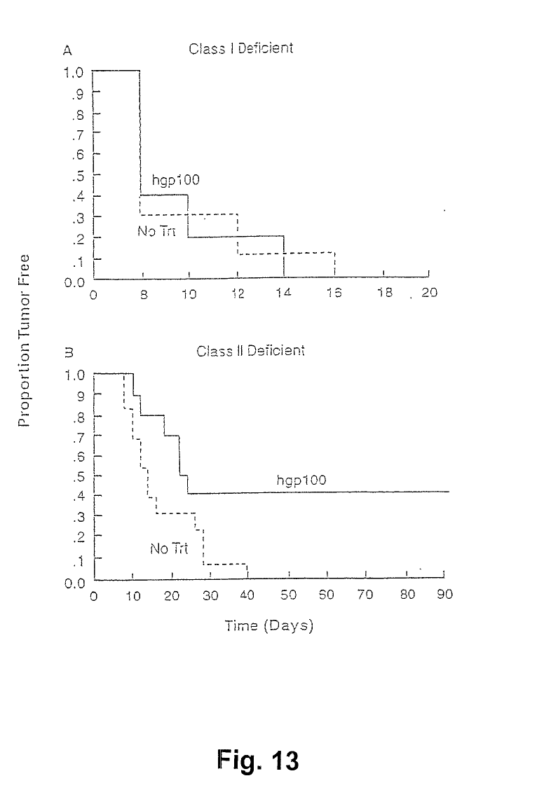

The RMA-S stabilization assays for MHC class I (FIGS. 11A-11H) demonstrate the binding of these six peptide pairs. Three peptide pairs including gp100₂₅₋₃₃ showed differential binding affinities with the human peptide being a stronger binder. T-cells from mice immunized with hgp100 DNA were tested for the presence of mouse peptide-specific CTL response by ⁵¹Cr release. Specific CTL response was detected only against the mgp 100₂₅₋₃₃ peptide (FIGS. 12A-12C). CTL response was detected after immunization with hgp100, but not syngeneic mgp100 or vector treated mice. Similarly, mice immunized with hgp100 DNA had significantly greater numbers of CD8⁺ T-cells responding to mgp 100₂₅₋₃₃ compared to mice immunized with mgp 100 DNA or control vector by Elispot assay for IFN-γ (FIGS. 13A and 13B). For the Elispot assay, lymphocytes were isolated from each mouse by preparing a single cell suspension from the draining inguinal lymph nodes and spleen. 5×10⁷ cells were cultured with 2×10⁷ naive irradiated (3,000 rads) splenocytes previously pulsed for one hour with 10 mM peptide. At five days, CD8⁺ T-cells were positively selected and used in an Elispot assay as previously described. Lewis, J. J., Int. J. Cancer 2000 (in press). 250,000 CD8⁺ cells were plated in each well of a 96 well plate and 5×10 peptide-pulsed EL4 cells were used per well as targets. Spot development was performed as described (Herr, W., J Infect Dis, 178(1):260-265 (1998)) and spots were counted using a stereomicroscope and automated computer (Zeiss, Germany) counting at 40 fold magnification. Experimental groups were plated in triplicate wells, and the means for different groups were compared by Student's t-test.

Immunization of mice with both hgp100 and mgp100 DNA was effective in generating antibodies against a human melanoma line expressing hgp100 (11 of 12 mice), but only hgp100 DNA resulted in occasional development of low-titer antibodies against the mouse melanoma line (2 of 6 mice). We have no explanation at this time why syngeneic mgp100 was able to induce antibodies that were specific for only the human melanoma cell line.

### EXAMPLE 15

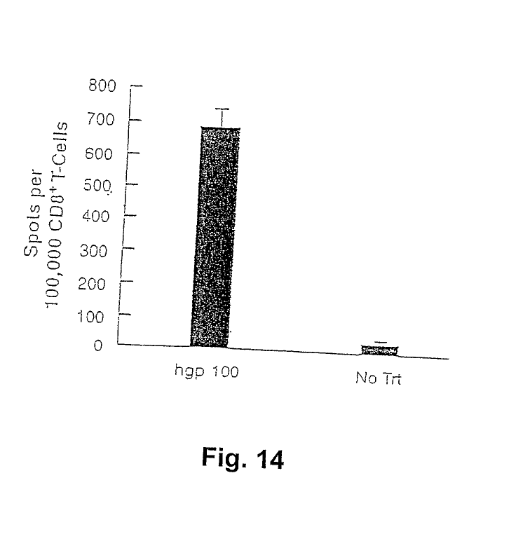

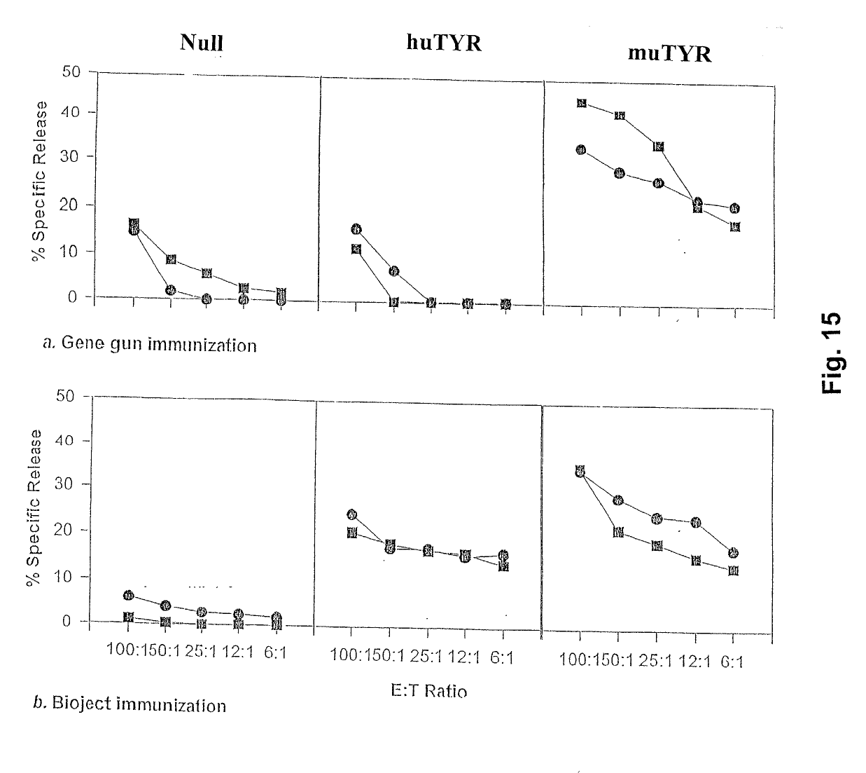

Tumor Protection is Dependent on CD8⁺ but not CD4⁺ T-cells. The role of different effector cell types in the antitumor response of hgp100 was investigated. μ2m −/− mice deficient in MHC class I and CD8⁺ T-cells, MHC II −/− mice deficient in MHC class II and CD4 ⁺ T-cells, and Igμ −/− mice deficient in mature B-cells were immunized with hgp100 or left untreated and subsequently challenged intradermally with B16. While MHC class I was required for tumor rejection, MHC class II was not, showing a role for CD 8⁺ T-cells but not CD4⁺ T-cells in tumor immunity resulting from immunization with hgp100 (FIGS. 14A and 14B). MHC class II deficient mice immunized with hgp100 also showed a CD8⁺ T-cell specific response against mgp100 as measured by IFN-γ release against mgp100₂₅₋₃₃ in an Elispot assay (FIG. 15). As reported previously, B-cell deficient mice have enhanced T-cell dependent tumor immunity. Browne, W. B., J Exp Med, 190(11):1717-1722 (1999); Qin, Z., Nat Med, 4(5):627-630 (1998). In our experience all B-cell deficient mice, either immunized or untreated, rejected B 16 melanoma after intradermal tumor challenge.

### EXAMPLE 16

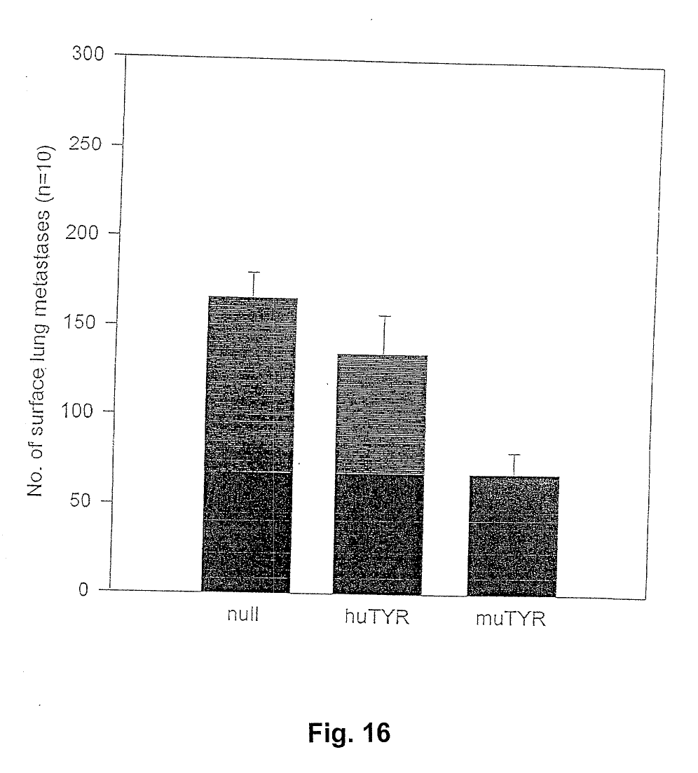

Cytotoxic T cell responses to murine tyrosinase (muTYR) in mice that were not immunized (null) or immunized with human tyrosinase (huTYR) or muTYR were evaluated. C57BL/6 mice, 2 per groups, were immunized cutaneously with DNA either by particle bombardment (gene gun) or needle-free jet injection (Bioject). Mice were immunized twice one week (FIG. 16A) or 10 days apart (FIG. 16B) with either 4 μg (FIG. 16A) or 100 μg (FIG. 16B) of DNA. 7 days after the last immunization, splenocytes were harvested and co-cultured with mitomycin C treated, con A activated mouse melanoma B16 cells for 5 days. Cytotoxicity against con A activated B16 target cells was then determined by a 4-hour standard ⁵¹Cr release assay.

### EXAMPLE 17

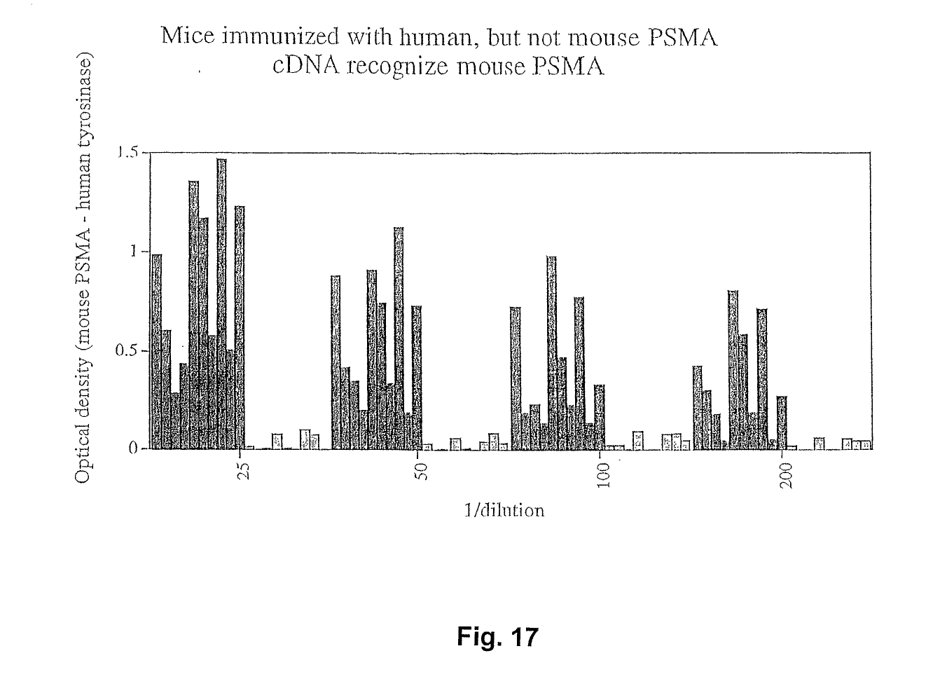

Protection and rejection of mouse melanoma B16F10/LM3 after immunization with mouse tyrosinase (muTYR) but not human tyrosinase (huTYR) DNA were evaluated. C57BL/6 mice, 10 per group, were immunized cutaneously weekly for 5 weeks with vector DNA (null), huTYR or muTYR DNA by particle bombardment. Two weeks after the last immunizations, mice were challenged with B16 melanoma cells intravenously and scored for surface lung metastases after 11-14 days. Significant tumor protection was observed in mice immunized with muTYR when compared with the null group (P<0.0001) or with mice immunized with huTYR (P=0.0153) (FIG. 17). Control null vector gave 165±14 rnetastases, huTYR gave 135±23, and muTYR gave 67±12 metastases. Results are shown as mean number of lung metastases ±SEM.

### EXAMPLE 18

Xenogeneic huTYR DNA vaccination induces self and autoantibodies. C57BL/6 mice, 10 per group, were immunized cutaneously with DNA either by particle bombardment (GG) or needle-free jet injection (BJ). Mice were immunized weekly for 5 weeks with either 4 μg (GG) or every 10 days with 100 μg (BJ) of DNA. Sera were collected from individual mice immunized with huTYR or muTYR after the last immunization. Antibody responses to xenogeneic human tyrosinase or syngeneic mouse tyrosinase were measured by immunoprecipitation. Human melanoma cells (Gmel) or mouse melanoma cells (B16) were lysed and followed by immunoprecipitation with either the positive control antibodies, pep7h/pep7 (a rabbit polyclonal antisera raised against the carboxyl-terminal peptide of huTYR/muTYR), or preimmune mouse sera or the test sera. The immunoprecipitated protein was then incubated in the substrate L-DOFPA which would result in the formation of melanin if tyrosinase was present. Results (Table 4) show the number of mice positive for antibody production.

TABLE 4

| Antigenic | | Melanin | No. of react- |
|---|---|---|---|
| Determinant | Source of Antibody | Formation | ive mice |
| human tyros- | pep7h (+control) | + | |
| inase (Gmel) | | | |
| | Pre-immune sera (−control) | − | |
| | Serum from mice immunized | | |
| | with: | | |
| | huTYR (GG) | + | 10/10 |
| | muTYR (GG) | − | 1/10 |
| | huTYR (BJ) | + | 9/10 |
| | muTYR (BJ) | − | 0/10 |
| mouse tyros- | pep7 (+control) | + | |
| inase (B16) | | | |
| | Pre-immune sera (−control) | − | |
| | Serum from mice immunized | | |
| | with: | | |
| | huTYR (GG) | + | 8/10 |
| | muTYR (GG) | − | 0/10 |
| | huTYR (BJ) | + | 9/10 |
| | muTYR (BJ) | − | 0/10 |

### EXAMPLE 19

Xenogeneic immunization against prostate specific antigens was analyzed. Mice were immunized 5 times by gene gun with the full-length human prostate specific membrane antigen (PSMA) cDNA (shaded bars, n=10) or with the full-length mouse PSMA cDNA (open bars, n=9). Sera from individual mice were screened by ELISA using plates coated with recombinant mouse PSMA protein, or as a control, recombinant human tyrosinase. The data are calculated by subtracting the optical density on tyrosinase-coated wells (approx. 0.34 OD units) from that of mouse PSMA-coated wells (FIG. 18).

We claim:

1. A method for stimulating an immune response to a tissue expressing a target differentiation antigen in a subject individual of a first species, comprising administering to the subject individual an immunologically-effective amount of a therapeutic differentiation antigen of the same type as the target differentiation antigen, wherein the therapeutic differentiation antigen is produced by expression in cells of a second species different from the first species.

2. The method according to claim 1, wherein the subject individual of the first species is human.

3. The method according to claim 1, wherein the target differentiation antigen is selected from the group consisting of Melan-A/MART-1, Pmel17, tyrosinase, gp75, gp100, CD19, CD20/CD20 B lymphocyte differentiation markers, muc-1, her2/neu, prostate specific antigen, prostatic acid phosphatase, and prostate specific membrane antigen.

4. The method according to claim 3, wherein the therapeutic differentiation antigen is expressed in insect cells.

5. The method of claim 2, wherein the step of administering is achieved by immunization with a vaccine comprising purified therapeutic differentiation antigen.

6. The method of claim 5, wherein the vaccine also comprises an adjuvant.

7. The method of claim 6, wherein the vaccine also comprises one or more cytokines.

8. The method of claim 2, wherein the step of administering is achieved by immunization with liposomes comprising purified therapeutic differentiation antigen.

9. The method according to claim 2, wherein the therapeutic differentiation antigen is a non-human differentiation antigen.

10. The method according to claim 9, wherein the therapeutic differentiation antigen is a mouse differentiation antigen.

11. The method according to claim 2, wherein the target differentiation antigen is expressed in melanocytes of the subject individual.

12. The method according to claim 11, wherein the therapeutic differentiation antigen is a human differentiation antigen.

13. The method according to claim 12, wherein the therapeutic differentiation antigen is expressed in insect cells.

14. The method according to claim 11, wherein the therapeutic differentiation antigen is a non-human differentiation antigen.

15. The method according to claim 14, wherein the therapeutic differentiation antigen is a mouse differentiation antigen.

16. A method for stimulating an immune response to a tissue expressing a target differentiation antigen in a subject individual of a first species, comprising administering to the subject individual an immunologically-effective amount of a therapeutic differentiation antigen of the same type derived from a second species different from the first species.

17. The method of claim 16, wherein the step of administering is achieved by immunization with a vaccine comprising purified therapeutic differentiation antigen.

18. The method of claim 17, wherein the vaccine comprises liposomes containing purified therapeutic differentiation antigen.

19. The method of claim 16, wherein the step of administering is achieved by immunization with DNA encoding the therapeutic differentiation antigen.

20. The method of claim 19, wherein the DNA immunization is achieved by immunization with liposomes comprising DNA encoding the therapeutic differentiation antigen.

21. The method of claim 19, wherein the DNA immunization is achieved by immunization with gold particles coated with DNA encoding the therapeutic differentiation antigen.

22. A method for stimulating an immune response to a tissue expressing a target differentiation antigen in a subject individual of a first species, comprising removing blood or bone marrow-derived cells from the subject individual, administering to the blood or bone marrow-derived cells an immunologically-effective amount of DNA encoding a therapeutic differentiation antigen of the same type derived from a second species different from the first species, and reintroducing the treated cells back into the subject individual.

23. The method of claim 22, wherein the step of administering is selected from the group consisting of liposomal transfection, particle bombardment and viral infection.

24. A method for stimulating an immune response to a tissue expressing a target differentiation antigen in a subject individual of a first species, comprising administering to the subject individual an immunologically-effective amount of a therapeutic differentiation antigen of the same type as the target differentiation antigen, wherein the therapeutic differentiation antigen is a mutant form of the target differentiation antigen.

25. The method of claim 24 wherein the mutant is a glycosylation mutant.

26. The method of claim 24, wherein the step of administering is achieved by DNA immunization.

27. The method of claim 26, wherein the mutant is a glycosylation mutant.

28. The method of claim 24, wherein the step of administering is achieved by immunization with a vaccine comprising purified therapeutic differentiation antigen.
